(19) United States
(12) Patent Application Publication
(10) Pub. No.: US 2009/0214426 A1
(43) Pub. Date: Aug. 27, 2009

(54) Assay Method

(76) Inventors: Laura Corradini, Kent (GB); Mark John Field, Kent (GB)

Correspondence Address: PFIZER INC.;PATENT DEPARTMENT, Bld 114 M/S 114, EASTERN POINT ROAD, GROTON, CT, 06340, US

(21) Appl. No.: 11/569,334

(22) PCT Filed: May 10, 2005
(86) PCT No.: PCT/IB2005/001403

§ 371 Date: Nov. 10, 2008

Related U.S. Application Data
(60) Provisional application No. 60/588,261, filed on Jul. 14, 2004.

(30) Foreign Application Priority Data
May 19, 2004 (GB) 0411140.7

(51) Int. Cl. A61K 49/00

(52) U.S. Cl. 424/9.1

(57) ABSTRACT

The present invention is related to the field of pharmacy. The present invention more particularly discloses a method of determining the emotional, sensory, physiological, social and cognitive effects of a pain condition in a non human animal and further discloses a method for preclinically identifying pharmaceutical therapeutics which improve the emotional, sensory, physiological, social and cognitive effects associated with a pain condition in a non human animal.

This invention relates to a method for the assessment of clinically relevant functional/quality of life (emotional, sensory, physiological, social and cognitive) deficit induced by pain in a non human animal and further relates to a method for preclinical identification of a pharmaceutical therapeutic which improves clinically relevant functional/quality of life measures in a non-human animal.

### BACKGROUND TO THE INVENTION

Persistent pain affects millions of people around the globe sometimes producing a chronic disease state that dramatically reduces the quality of life (QoL) of patients. Quality of life encompasses emotional, sensory, physiological, psychological, social and cognitive functions. People enduring long lasting pain, such as for example chronic neuropathic pain, often experience disruption to a variety of functional aspects of their normal daily life and find that their pain condition has pronounced affects on the physiological, emotional, social and cognitive domains of their life (Niv and Kreitler, 2001; Skevington et al, 1998). A consequence of pain is often that such aspects of the quality of life of patients is severely compromised, and it has been found that it is very hard to restore it with current analgesic therapies. The complexity of such pain associated debilitation is reflected by the lack of instruments for measuring QoL in chronic pain conditions. The 9^{th} World Congress on Pain (Vienna, August 1999) concluded that there is an unmet medical need for the treatment of pain and particularly chronic pain. It is therefore desirable that a therapeutic strategy for pain should consider this broader picture of the condition in order to develop pharmaceutical compounds able to relieve pain and its associated functional effects or functional deficits and restore the quality of life (QOL) of patients to normal levels. The limited number of clinical studies, where QoL has been assessed, has not provided sufficient data to prove the efficacy of current analgesics in restoring normal functionality of patients. Randomised controlled clinical trials using the anti epileptic gabapentin demonstrated an improvement in the QoL of patients with diabetic neuropathy and post herpetic neuralgia (Backonja M et al, 1998; Rowbotham M et al, 1998). Similar randomised controlled clinical trials using Pregabalin showed efficacy in treating the QoL deficit of fibromyalgia patients (Mease P J et al, 2003). No other compound has a similar profile in clinical protocols, indicating that current methods of pre clinical behavioural tests for non human animals, used in the process of selection of pharmaceutical compounds, are incomplete and do not guarantee a selection of medicines with a globally acting efficacy in humans for numbers of symptoms associated with pain conditions.

The effects of pain, and particularly chronic pain, are difficult to identify preclinically due to the above mentioned multiple pain symptoms which can encompass the emotional, sensory, physiological, social and cognitive functions of the effected subject.

In the recent European neuropathic Pain Conference in Madrid 2004 it was stated that there was the need to improve animal models to better understand the variability in causes and symptoms observed in patients. Therefore the development of pre-clinical models of pain conditions that reflect the clinical assessments seen in human subjects are important to providing a medical understanding of the pathophysiology of pain and it's associated impact on quality of life effects; also in helping to identify new treatments for this condition, particularly treatments that effect the physical pain threshold and the quality of life experienced by the subject. Hence it is desirable to be able to provide a method for assessing the QoL effects, the emotional, sensory, physiological, social and cognitive, associated with a pain condition in a non human animal. It is further desirable to provide a method for identifying pharmaceutical compounds which effect the emotional, sensory, physiological, social and cognitive effects associated with a pain condition in a non human animal.

There are known behavioural studies in rodents which are directed to the assessment of animal welfare (Schrijver N C et al, 2002), also Bauhofer and colleagues (2002) explored sickness behaviour in septic rats. Schoemaker et al (1996) measured the behaviour in rats with myocardial infarction after pharmacological treatment and found results in agreement with clinical trials. However little interest has been shown in assessing the effects of pain conditions on behaviour and quality of life, Kontinen et al, 1999 have developed rodent models of neuropathic pain and have reported negative observations indicating that there were no behavioural differences between nerve injured and sham-operated rats.
1. We have demonstrated that animal models of specific pain conditions develop not only changes in the pain-like threshold but also pain-related physical deficit, mood and cognitive disorders which correlate well with those observed in humans with chronic pain disease (McCracken L M et al, 1998; Gureje 0 et al, 1998 JAMA; 280:147-151; MacWilliams L A et al 2003 Pain, 106:127-33; Apkarian A V et al, 2004).

### BRIEF DESCRIPTION OF THE INVENTION

The invention makes available a method of determining the emotional, sensory, physiological, social and cognitive effects of a pain condition in a non human animal. The method has the advantage that it provides a validity model of a specific pain condition and allows the fuller investigation of the complex picture of pain in a way that correlates well with clinical observations. The present invention further makes available a method for identifying pharmaceutical compounds that effect the emotional, sensory, physiological, social and cognitive effects associated with a pain condition in a non human animal. The method has the advantage that it allows the identification of compounds with efficacy in restoring functionality and QoL. Further the methods of the present invention includes steps to ensure that the behaviour measured is specifically pain related i.e. related solely to the intended pain condition, and that steps employed to assess the QoL avert the evoking of stress in the animal subject such that the behavioural measures are essentially free from artefacts auxiliary to the intended behaviour measured.

The present invention makes it possible to compare the broader human domains of pain with animal's behaviour using the methods as instruments for the assessment of secondary measures in rodents with pain conditions such as a chronic pain disease.

### DETAILED DESCRIPTION OF THE INVENTION

In its first and broadest aspect the present invention provides an assay comprising the following steps:

(a) providing a non human test animal which has been arranged to experience a pain condition,
(b) determining the degree to which the test animal experiences pain,
(c) determining whether the test animal experiences pain or physical impairment not associated with the pain condition and selecting the test animal if the pain is associated predominantly with the pain condition,
(d) determining a performance score for the selected test animal in one or more behavioural tests,
(e) determining whether the selected test animal demonstrates an increase, decrease or no change in performance score compared to the performance score obtained for a control animal.

2. A modification of the assay of aspect 1 comprising the following steps:

(a) providing a non human test animal which has been arranged to experience a pain condition,
(b) determining the degree to which the test animal experiences pain,
(c) determining whether the test animal experiences pain or physical impairment not associated with the pain condition and selecting the test animal if the pain is associated only with the pain condition,
(d) determining the performance score for the selected test animal in one or more behavioural tests.
(e) administering a test compound to the selected test animal, preferably the selected animal also demonstrates a poor performance score with respect to a control animal in the behavioural test,
(f) determining whether there is an increase, decrease or no change in the degree to which the selected test animal continues to experience pain,
(g) redetermining a performance score for the selected test animal in the one or more behavioural tests,
(h) determining whether the selected test animal demonstrates an increase, decrease or no change in performance score.

The non human animal may be a vertebrate, for example a mammal, amphibian, reptile and bird; preferably the animal may be a mammal such as a mouse, a rat and other rodents, a pig, a cow, a bull, a sheep, a horse, a dog or a rabbit or any farmed animal, more preferably the animal may be a mouse or a rat, most preferably a rat.

The pain condition may be any physiological pain such as inflammatory pain or nociceptive pain or neuropathic pain or acute pain or chronic pain, musculo-skeletal pain, on-going pain, central pain, heart and vascular pain, head pain, orofacial pain; preferably it is neuropathic pain. Other pain conditions include intense acute pain and chronic pain conditions which may involve the same pain pathways driven by pathophysiological processes and as such cease to provide a protective mechanism and instead contribute to debilitating symptoms associated with a wide range of disease states. Pain is a feature of many trauma and disease states. When a substantial injury, via disease or trauma, to body tissue occurs the characteristics of nociceptor activation are altered. There is sensitisation in the periphery, locally around the injury and centrally where the nociceptors terminate. This leads to hypersensitivity at the site of damage and in nearby normal tissue. In acute pain these mechanisms can be useful and allow for the repair processes to take place and the hypersensitivity returns to normal once the injury has healed. However, in many chronic pain states, the hypersensitivity far outlasts the healing process and is normally due to nervous system injury. This injury often leads to maladaptation of the afferent fibres (Woolf & Salter 2000 Science 288: 1765-1768). Clinical pain is present when discomfort and abnormal sensitivity feature among the patient's symptoms. Patients tend to be quite heterogeneous and may present with various pain symptoms. There are a number of typical pain subtypes: 1) spontaneous pain which may be dull, burning, or stabbing; 2) pain responses to noxious stimuli are exaggerated (hyperalgesia); 3) pain is produced by normally innocuous stimuli (allodynia) (Meyer et al., 1994 Textbook of Pain 13-44). Although patients with back pain, arthritis pain, CNS trauma, or neuropathic pain may have similar symptoms, the underlying mechanisms are different and, therefore, may require different treatment strategies. Therefore pain can be divided into a number of different areas because of differing pathophysiology, these include nociceptive, inflammatory, neuropathic pain etc. It should be noted that some types of pain have multiple aetiologies and thus can be classified in more than one area, e.g. Back pain, Cancer pain have both nociceptive and neuropathic components.

Nociceptive pain is induced by tissue injury or by intense stimuli with the potential to cause injury. Pain afferents are activated by transduction of stimuli by nociceptors at the site of injury and sensitise the spinal cord at the level of their termination. This is then relayed up the spinal tracts to the brain where pain is perceived (Meyer et al., 1994 Textbook of Pain 13-44). The activation of nociceptors activates two types of afferent nerve fibres. Myelinated A-delta fibres transmitted rapidly and are responsible for the sharp and stabbing pain sensations, whilst unmyelinated C fibres transmit at a slower rate and convey the dull or aching pain. Moderate to severe acute nociceptive pain is a prominent feature of, but is not limited to pain from strains/sprains, post-operative pain (pain following any type of surgical procedure), posttraumatic pain, burns, myocardial infarction, acute pancreatitis, and renal colic. Also cancer related acute pain syndromes commonly due to therapeutic interactions such as chemotherapy toxicity, immunotherapy, hormonal therapy and radiotherapy. Moderate to severe acute nociceptive pain is a prominent feature of, but is not limited to, cancer pain which may be tumour related pain, (e.g. bone pain, headache and facial pain, viscera pain) or associated with cancer therapy (e.g. postchemotherapy syndromes, chronic postsurgical pain syndromes, post radiation syndromes), back pain which may be due to herniated or ruptured intervertabral discs or abnormalities of the lumber facet joints, sacroiliac joints, paraspinal muscles or the posterior longitudinal ligament.

Neuropathic pain is defined as pain initiated or caused by a primary lesion or dysfunction in the nervous system (IASP definition). Nerve damage can be caused by trauma and disease and thus the term ‘neuropathic pain’ encompasses many disorders with diverse aetiologies. These include but are not limited to, Diabetic neuropathy, Post herpetic neuralgia, Back pain, Cancer neuropathy, HIV neuropathy, Phantom limb pain, Carpal Tunnel Syndrome, chronic alcoholism, hypothyroidism, trigeminal neuralgia, uremia, or vitamin deficiencies. Neuropathic pain is pathological as it has no protective role. It is often present well after the original cause has dissipated, commonly lasting for years, significantly decreasing a patients quality of life (Woolf and Mannion 1999 Lancet 353: 1959-1964). The symptoms of neuropathic pain are difficult to treat, as they are often heterogeneous even between patients with the same disease (Woolf & Decosterd 1999 Pain Supp. 6: S141-S147; Woolf and Mannion 1999 Lancet 353: 1959-1964). They include spontaneous pain, which can be continuous, or paroxysmal and abnormal evoked pain, such as hyperalgesia (increased sensitivity to a noxious stimulus) and allodynia (sensitivity to a normally innocuous stimulus).

The inflammatory process is a complex series of biochemical and cellular events activated in response to tissue injury or the presence of foreign substances, which result in swelling and pain (Levine and Taiwo 1994: Textbook of Pain 45-56). Arthritic pain makes up the majority of the inflammatory pain population. Rheumatoid disease is one of the commonest chronic inflammatory conditions in developed countries and rheumatoid arthritis is a common cause of disability. The exact aetiology of RA is unknown, but current hypotheses suggest that both genetic and microbiological factors may be important (Grennan & Jayson 1994 Textbook of Pain 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder 2002 Ann Pharmacother. 36: 679-686; McCarthy et al., 1994 Textbook of Pain 387-395). Most patients with OA seek medical attention because of pain. Arthritis has a significant impact on psychosocial and physical function and is known to be the leading cause of disability in later life. Inflammatory pain thus includes arthritic pain, including pain resulting from osteoarthritis and rheumatoid arthritis, other types of inflammatory pain include but are not limited to inflammatory bowel diseases (IBD),

Other types of pain include but are not limited to;
Musculo-skeletal disorders including but not limited to myalgia, fibromyalgia, spondylitis, sero-negative (non-rheumatoid) arthropathies, non-articular rheumatism, dystrophinopathy, Glycogenolysis, polymyositis, pyomyositis.
Central pain or ‘thalamic pain’ as defined by pain caused by lesion or dysfunction of the nervous system including but not limited to central post-stroke pain, multiple sclerosis, spinal cord injury, Parkinson's disease and epilepsy.
Heart and vascular pain including but not limited to angina, myocardical infarction, mitral stenosis, pericarditis, Raynaud's phenomenon, scleredoma, scleredoma, skeletal muscle ischemia.
Visceral pain, and gastrointestinal disorders. The viscera encompasses the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders include the functional bowel disorders (FBD) and the inflammatory bowel diseases (IBD). These GI disorders include a wide range of disease states that are currently only moderately controlled, including—for FBD, gastro-esophageal reflux, dyspepsia, the irritable bowel syndrome (IBS) and functional abdominal pain syndrome (FAPS), and—for IBD, Crohn's disease, ileitis, and ulcerative colitis, which all regularly produce visceral pain. Other types of visceral pain include the pain associated with dysmenorrhea, pelvic pain, cystitis and pancreatitis.

Head pain including but not limited to migraine, migraine with aura, migraine without aura cluster headache, tension-type headache. Orofacial pain including but not limited to dental pain, temporomandibular myofascial pain, tinnitus, hot flushes, restless leg syndrome and blocking development of abuse potential. Further pain conditions may include, back pain, bursitis, dental pain, fibromyalgia or myofacial pain, menstrual pain, migraine, neuropathic pain (including painful diabetic neuropathy), pain associated with post-herpetic neuralgia, post-operative pain, referred pain, trigeminal neuralgia, visceral pain (including interstitial cystitis and IBS) and pain associated with AIDS, allodynia, burns, cancer, hyperalgesia, hypersensitisation, spinal trauma and/or degeneration and stroke.

The animal may be arranged to experience a pain condition by surgical intervention by to cause a physical lesion or damage by a surgical procedure on the animal, preferably the procedure involves damage to a peripheral nerve for example by use of the Bennett model, loose chromic ligature of the sciatic nerve, (Bennett, G. J. (1994) Neuropathic Pain, in Text book of Pain; Wall, P. D. and Meizack, R., eds; pp. 201-224, Churchill Livingstone), or of the Seltzer model, partial tight ligation of the sciatic nerve (Seltzer, Z. (1995) The relevance of animal neuropathy models for chronic pain in humans. Sem. Neurosci, 8: pp. 34-39) or of Chung's model, tight ligation of one of the two spinal nerves of the sciatic nerve (Kim S H, Chung J M. An experimental model for peripheral neuropathy produced by segmental spinal nerve ligation in the rat. Pain (1992); 50: pp. 355-63) or of the Chronic Constriction Injury model (CCI) (Bennett G J, Xie Y-K. A peripheral mononeuropathy in rat that produces disorders of pain sensation like those seen in man. Pain (1988); 33: pp. 87-107) or any other peripheral nerve injury method.

The animal may alternatively be arranged to experience a pain condition by administration of pain inducing agent, for example Capsaicin (Dirks J, Petersen K L, Rowbotham M C, Dahl J B. Gabapentin suppresses cutaneous hyperalgesia following heat-capsaicin sensitisation, Anesthesiology. 2002 July; 97(1): pp. 102-107) or Formalin (Tjolsen, A. et. al (1992) The Formalin Test, an evaluation of the method, Pain 51, pp. 5-17) or Freunds Complete Adjuvant (Abdi seconds, Vilassova N, Decosterd I, et al. Effects of KRN5500, a spicamycin derivative, on neuropathic and nociceptive pain models in rats. Anesth Analg 2000; 91: pp. 955-99) or Carrageenan (Itoh, M., Takasaki, I., Andoh, T., Nojima, H., Tominaga, M. & Kuraishi, Y. (2001)

Induction by carrageenan inflammation of prepronociceptin mRNA in VR1-immunoreactive neurons in rat dorsal root ganglia. Neurosci. Res., 40, pp. 227-233) or Taxol (Polomano R C. Mannes A J. Clark U S. Bennett G J. A painful peripheral neuropathy in the rat produced by the chemotherapeutic drug, paclitaxel. (2001) Pain. 94(3): pp. 293-304) or vinca alkaloid, vincristine (Aley K O, Reichling D B, Levine J D. Vincristine hyperalgesia in the rat: a model of painful vincristine neuropathy in humans. Neuroscience (1996); 73: pp. 259-65) or Turpentine (Ness T J, Gebhart G F. Visceral pain: a review of experimental studies. Pain (1990); 41: pp. 167-234 and McMahon S B. Neuronal and behavioral consequences of chemical inflammation of rat urinary bladder. Agents Actions (1988); 25: pp. 231-233).

Alternatively the animal may be arranged to experience a pain condition by providing to the animal a noxious physical stimulus, for example by administration of noxious heat stimulus (Malmberg, A. B., and Bannon, A. W. Models of nociception: hot-plate, tail-flick, and formalin tests in rodents. Current Protocols in Neuroscience 1999; pp 8.9.1-8.9.15) or by administration of noxious cold stimulus or noxious pressure stimulus or UV-irradiation (seconds. J. Boxall, A. Berthele, D. J. Laurie, B. Sommer, W. Zieglgänsberger, L. Urban and T. R. Tölle, Enhanced expression of metabotropic glutamate receptor 3 messenger RNA in the rat spinal cord during ultraviolet irradiation induced peripheral inflammation Neuroscience (1998) 82(2): pp. 591-602).

Alternatively the animal may be arranged to experience a pain condition by a process of selection to select an animal that naturally possesses a painful disease condition such as arthritis or HIV or Herpes or cancer or diabetes. Alternatively the animal may be arranged to experience pain by modification of the animal to possess a painful disease condition such as arthritis or HIV or Herpes or cancer or diabetes. Animals may be modified to possess a painful disease in a variety of ways for example by administration of Streptozocin to induce a diabetic neuropathy (Courteix, C., Eschalier, A., Lavarenne, J., Streptozocin-induced diabetic rats: behavioural evidence for a model of chronic pain, Pain, 53 (1993) pp. 81-88) or by administration of viral proteins to cause HIV related neuropathic pain (Herzberg U. Sagen J. Peripheral nerve exposure to HIV viral envelope protein gp120 induces neuropathic pain and spinal gliosis. Journal of Neuroimmunology. (2001 May 1), 116(1): pp. 29-39) or administration of Complete Freunds Adjuvant or Mono-iodoacetate to induce arthritis and inflammatory pain (Rikard Holmdahl, Johnny C. Lorentzen, Shemin Lu, Peter Olofsson, Lena Wester, Jens Holmberg, Ulf Pettersson Immunological Reviews Arthritis induced in rats with non-immunogenic adjuvants as models for rheumatoid arthritis (2001) Volume 184, Issue 1, pp. 184) or administration of varicella zoster virus to cause Herpes and post herpatic neuralgia (Fleetwood-Walker S M. Quinn J P. Wallace C. Blackburn-Munro G. Kelly B G. Fiskerstrand C E. Nash A A. Dalziel R G. Behavioural changes in the rat following infection with varicella-zoster virus. Journal of General Virology. 80 (Pt 9):2433-6, 1999 September) or administration of a carcinogen or of cancer cells to an animal to cause cancer (Shimoyama M. Tanaka K. Hasue F. Shimoyama N. A mouse model of neuropathic cancer pain, Pain. 99(1-2): pp. 167-74, 2002 September). Preferably the animal is arranged to experience a pain condition using the Chronic Constrictive Injury model (CCI) preferably as described in the examples below.

The animal in step (a) aspect 1 and step (a) aspect 2 can be arranged to experience the pain condition using the same method.

It is not critical which method is used to determine the degree to which an animal experiences pain in step (b) of aspect 1 or in steps (b) and (f) of aspect 2, it is advisable that the same method be used for step (b) and (f) of aspect 2. A variety of methods may be used; for example the degree to which the animal experiences pain is determined by use of any suitable method for measurement of a pain threshold deficit, for example a Von Frey test for assessment of tactile mechanical allodynia (Chaplan S R, Bach F W, Pogrel J W. Quantitative assessment of allodynia in the rat paw. J Neurosci Methods 1994; 53: pp. 55-63) or Paw withdrawal test (Tal M, Bennett G. Extra-territorial pain in rats with a peripheral mononeuropathy: mechano-hyperalgesia and mechano-allodynia in the territory of an uninjured nerve. Pain 1994; 57: pp. 375-82) or Pinprick hyperalgesia test (Koltzenburg M. Painful neuropathies. Curr Opin Neurol 1998; 11: pp. 515-21) or Hotplate test (Nishiyama T, Yaksh T L, Weber E. Effects of intrathecal NMDA and non-NMDA antagonists on acute thermal nociception and their interaction with morphine. Anesthesiology 1998; 89: pp. 715-22) or Thermal allodynia test (Bennett G. Neuropathic Pain. In: Wall P D, Melzack R, eds. Textbook of pain. Edinburgh: Churchill Livingstone, 1994: pp. 201-24) or Dynamic allodynia test (Koltzenburg M, Torebjork E, Wahren L K. Nociceptor modulated central sensitization causes mechanical hyperalgesia in acute chemogenic and chronic neuropathic pain. Brain 1994; 117: pp. 579-91) or Thermal hyperalgesia test (Hargreaves K M, Dubner R, Brown F, Flores C, Joris J: A new and sensitive method for measuring thermal nociception in cutaneous hyperalgesia. Pain 1988; 32: pp. 77-88) or Noxious heat or cold pain pressure pain tests (Courteix, C., Eschalier, A., Lavarenne, J., Streptozocin-induced diabetic rats: behavioural evidence for a model of chronic pain, Pain, 53 (1993) pp. 81-88) or weight bearing test (Schott et al., 1994 Journ. Pain. Man. 31: pp. 79-83); preferably the degree to which the animal experiences pain is determined by the degree to which the animal is able to support its body weight using a weight bearing test or by a Paw withdrawal test using a Von Frey hair to measure static allodynia and/or by a Paw withdrawal test using a cotton wool bud to measure dynamic allodynia. Most preferably a Paw withdrawal test using a Von Frey hair to measure static allodynia and/or a Paw withdrawal test using a cotton wool bud to measure dynamic allodynia are carried out.

The degree to which a test animal experiences pain according to step (b) of aspects 1 and 2, is preferably assessed by reference to the equivalent measurement for a control animal which is subjected to the same test as the test animal under the same conditions such that the resulting measures can be compared to extract a degree of the measured effect in the test animal relative to the control.

The degree to which a test animal continues to experience pain according to step (f) of aspect 2 is preferably assessed by comparison to a previous measurement obtained from the same test animal in the same test, for example that measurement made in step (b) of aspect 2, additionally or alternatively the comparison may be made to the equivalent test measurement for a control animal which is subjected to the same test as the test animal under the same conditions.

The term “control animal” as used herein is intended to include either naïve animals or sham animals. A naïve animal is preferably a normal healthy animal free from disease and particularly diseases or conditions linked to pain or to behavioural abnormality (for example neurodegenerative diseases or mood disorders or cognitive deficits) preferably the naïve animal does not show indications of an abnormal pain threshold when tested using known tests for pain (i.e. static and dynamic allodynia or weight bearing test). A sham animal is preferably a naïve animal which has been subjected to any intervention performed on the test animal, performed in the test animal in order to produce a pain condition but without providing the causative effect of the pain condition (for example where an injection is given in the test animal a placebo injection is given in the sham, or where an incision is made to ligature a nerve in a test animal the incision is made but the nerve is not ligatured in the sham). Preferably the sham animal and test animal are subjected to any relevant intervention at a similar point in time, for example on the same day.

The control animal is preferably the same species of animal as the test animal and is additionally preferably of essentially the same age, size, weight and the same sex.

Determining whether the animal experiences pain or physical impairment not associated with the pain condition can be carried out using a test such as a locomotor impairment test suitable for assessing locomotor impairment for example, direct observation, gait analysis test for motor co-ordination, grip strength test for muscle tone, pawslips test (Melnick S M et al, Pharmacol, bioch and behaviour, 72, 2002) for ataxia or the rota rod test, preferably the rota rod test is used. The test is preferably performed on the test animal to obtain a measurement of locomotor impairment in comparison to the equivalent measurement for a control animal which is subjected to the same test as the test animal under the same conditions. The control animal can be a naïve animal or a sham animal, preferably a naïve animal. In the test animal of aspects 1 and 2 the pain is taken to be associated predominantly with the pain condition if the measurement from the locomotor impairment test is within 20% of that expected or obtained from a sham operated or naïve animal, preferably within 10%, more preferably within 5% further preferably within 2%.

A behavioural test as performed in aspects 1 and 2 of the present invention is preferably designed to measure one or more of cognitive function, social well-being, emotional well-being or physiological well-being. Further preferably the behavioural test is designed to measure one or more of; learning ability, memory, social interaction, exploratory behaviour, motivation, anxiety, depression, spontaneous locomotion and activity, fear of movement (Kinesaphobia), sexual behaviour, quality of sleep, blood pressure, heart rate, change in body weight, general health. The behavioural test may be for example any of:
1 learning ability tests: which may be assessed by a maze learning test e.g. standard radial arm maze or T maze tests lined with a food reward, or by the shuttle-box test (evaluating the latency time and the number of errors for several days in succession).
2 sexual behaviour tests: which may be assessed by:
Measures of mounts, intromissions and ejaculations, recorded to assess sexual motivation and performance in male rats. For example:
   Mount Latency: the time that elapses between introducing the male and female to the test apparatus before the male mounts the female
   Intermount Interval: the average time between successive mounts
   Inter Intromission Interval: the average time between successive intromissions
   Post ejaculatory Interval: the time elapsing between an ejaculation and the next copulatory series.
Measures of female sexual behaviour include:
   Receptivity: the females willingness and ability to copulate—(lordosis score)
   Proceptivity: the females eagerness to copulate which can be assessed by measuring female hopping, darting, investigatory and presenting (adopting the lordosis posture) behaviours.
   Attractiveness: how willing are males to copulate with the female.
3 social interaction tests: which may be assessed by:
Monitoring the interaction of a test rat with a ‘stranger’ rat in an arena whereby the time the rats spends sniffing, grooming, each other is recorded.
4 exploratory behaviour tests: which may be assessed by a hole board test whereby the animal explores an arena with ‘holes’ in the floor to which it can place its head into to investigate it. The number of entries into the holes and number of holes explored are recorded.
5 motivation tests: which may be assessed by a hole board test as described above.
6 anxiety tests: which may be assessed by an elevated plus maze wherein an animal is introduced into a raised maze, above the floor, with 4 narrow arms arranged as a cross at right angles where two arms are enclosed and two arms are open. An anxiety measure is recorded when the animal will not enter the open arms.
7 depression tests: which may be assessed by a forced swim test, rats are placed in a chamber, filled with water, with no escape. The depressive state is measured by lack of swimming or trying to escape from chamber. An alternative test includes the tail suspension test where mice are suspended by the tail. The immobility time is measured, as an indicator of depression.
8 spontaneous locomotion and activity tests: which may be assessed by the measurement of horizontal and vertical locomotor activity in a novel environment, usually recorded by photocells-equipped cages or video tracking system.
9 memory, which may be assessed by
Morris watermaze test using a circular arena filled with water, visual clues placed around the arena and time spent finding the exit using the visual clues as reference point are recorded. An alternative test is novel object recognition test which consists of two phases. Rats are selected, in the first phase, for their ability to explore equally two identical objects in a familiar arena In the second phase, one object is replaced with a new one and animals are allowed to explore the arena freely again. The difference in exploration between novel and familiar object in the second phase is a measurement of a memory abilities.
10 sleep quality tests: which may be assessed by the analysis of EEG recordings from animals.

The behavioural test may also be for example, the locomotor activity test, preferably the locomotor activity test as described in the examples below, or the beam walking test, preferably the beam walking test as described in the examples below, or the rota rod test, preferably the rota rod test as described in the examples below, or the open field test, preferably the open field test as described in the examples below, or the object recognition test, preferably the object recognition test as described in the examples below. According to aspects 1 and 2 of the present invention one or more of the behavioural tests may be performed.

In the performance of the Beam walking test preferably the cut off time is set at 20 seconds (time) for those rats that fall off the beam or do not cross or freeze while are crossing the beam and a cut off 10 (foot slips) for those rats that fall off the beam, do not cross, freeze or do not use an injured paw (for example in nerve injured animals such as CCI animals) while are crossing the beam—this helps to reduce the variability in the data in order to quantify the physical dysfunction.

Dosing of animals with test compounds for the Beam Walking, Open Field and object recognition tests are preferably carried out in selected animals showing decreased pain threshold and functional impairments in the specific test: for example in the Beam Walking only those CCI rats showing a static allodynic pain-like threshold and a number of foot slips greater than 2 before the compound is administered at 2 weeks post surgery and for Open Field only those CCI rats showing an static allodynic pain-like threshold and a number of entries into the center of the arena less than or equal to 6 at 2 weeks post surgery or for object recognition test those CCI rats showing an static allodynic pain-like threshold and a discrimination index value±10 seconds in the first phase of the test at 2 weeks post surgery.

In the performance of the open field method the study is preferably carried out in normal light condition 60 Lux instead of bright light condition (high light intensity-induced anxiety in naïve rats) in order to reduce anxiety effects on the subject.

The term “performance score” as used herein is intended to include the measured quantity of a variable indicative of a particular behavioural function during the performance of a behavioural test, for example the performance score may be number of times an animal enters the central zone as a measure of the level of anxiety in the open field test, number of foot slips or time to cross in a beam walking test, time period of exploration in the object recognition test.

Determining a performance score for the selected test animal in one or more behavioural tests according to aspect 1 step (d) and aspect 2 steps (d) and (h) may be done in comparison to the equivalent measurement for a control animal which is subjected to the same test as the test animal under the same conditions.

The increase, decrease or lack of change in a) the degree to which an animal continues to experience pain or b) performance score in a behavioural test, may be judged by direct comparison between determinations made or by using a statistical analysis of the resultant measure or observation which is the output of the method used to determine a) the degree of pain experienced by an animal or b) the performance score in a behavioural test. Typically the statistical analysis enables the determination of whether an observed or measured quantity differs significantly from that quantity or range of quantity expected or measured in the absence of the test compound. The procedure may be any standard mathematical statistical procedure for assessment of statistical significance, for example; tests of hypotheses, tests of significance, rules of decision, or decision rules. Typically the level of significance, or significance level, of the selected statistical procedure, often denoted by a, is pre-specified, in practice, preferably a significance level of 0.05 or 0.01 is used, although other values may be used. If, for example, the 0.05 (or 5%) significance level is chosen in designing a decision rule for testing significance of a quantity, then there are about 5 chances in 100 of a rejection of the hypothesis that a quantity is insignificantly different from what would be expected for example in the absence, or in the presence, of a test compound when it should be accepted as significant; that is, there is a 95% confidence that the right decision has been made. In such case at the 0.05 significance level, the hypothesis has a 0.05 probability of being wrong. Critical values corresponding to α=0.05, 0.01, and 0.001 are tabulated for many commonly used statistics, such as those for the t-test, F-test and chi-squared test, and may be used in the assessment of judging significance. Typically a 0.001 to 0.05 significance level is used.

Where a quantity value is compared to a range of quantity values then significance is preferably judged by determination of the standard deviation of the quantity value from the mean of the distribution of the range of quantity values, typically a value of 2 or 3 standard deviations from the mean is taken as being significant, normalisation of the distribution may be necessary using standard procedures prior to calculation of the standard deviation.

The test compound according to aspect 2 of the present invention is preferably a pharmaceutical compound and can be delivered by any standard method for example orally or intravenously or injected parenterally or injected intramuscularly or injected subcutaneously or by inhalation or by suppository or pessary or topically, preferably the dose is delivered orally. The dose of a compound is typically of the range from 0.01 to 300 mg/kg body weight of the subject animal, preferably 0.1 to 100 mg/kg. Alternatively the dose may be delivered by intravenous infusion, preferably at a dose which of the range from 0.001-100 mg/kg/hr. The above dosages are exemplary of the average case and may be more or less in quantity accordingly.

According to aspect 3 of the present invention there is provided a modification of the second aspect of the invention wherein more than one test compound may be administered.

According to aspect 4 of the invention there is provided a pharmaceutical composition comprising the compound according to aspect 2.

According to aspect 5 of the invention there is provided a compound according to aspect 2 for use as a medicament.

According to aspect 6 of the invention there is provided the use of a compound according to aspect 2 in the preparation of a medicament for the treatment of a pain condition, preferably for the treatment of neuropathic pain.

According to aspect 7 of the invention there is provided a pharmaceutical composition comprising the combination test compounds according to aspect 4.

According to aspect 8 of the invention there is provided a combination according to aspect 4 for use as a medicament.

According to aspect 9 of the invention there is provided the use of a combination according to aspect 4 in the preparation of a medicament for the treatment of a pain condition, preferably for the treatment of neuropathic pain.

The following examples illustrate the embodiments and principles of the invention.

### EXAMPLES

The following examples demonstrate the successful investigation into the two main domains damaged by persistent pain, specifically the physiological, the emotional and cognitive domains as assessed through measures of physical impairment, emotional changes and memory dysfunction in rodents. Measures are made with care so as to avoid environmental stress that could affect the behavioural outcomes when measurements or scores are taken in the performance of any test as applied to both test and control animals (for example animals are preferably trained or habituated to various test conditions). The chronic constriction injury (CCI) rat model is used to provide a model of chronic neuropathic pain, behavioural motor, emotional and cognitive abnormalities. QoL measures are assessed using a variety of tests such as locomotor activity, rota rod, beam walking and open field tests and memory or learning tests such as an object recognition test.

CCI rats display allodynic-like behaviour, weight bearing deficit and beam walking impairments up to 6 weeks post nerve ligation. From the rota rod measurements, it seems that major physical impairment is displayed until 2 weeks post surgery. Starting from 4 weeks post surgery, the rota rod performance scores of CCI rats are comparable to naïve and sham-operated rats thus a locomotor impairment test such as the rota rod test allows the selection of animals not suffering from temporary physical impairment auxiliary to the induced pain condition.

Rats with CCI of the sciatic nerve show pain-related behavioural, motor, emotional and cognitive abnormalities. At two weeks post injury, CCI rats show a significant decrease in the pain-like threshold (p<0.01) and deficits in the locomotor activity, rota rod and beam walking tests (p<0.05 and p<0.01). However, at 4 weeks, when most of the motor functionality is restored, rats still display allodynic pain-like threshold. At this stage animals keep showing deficits in crossing the elevated beam, exploring the open field and in recognizing a novel object. These changes disappear at about 8 weeks post surgery when most of the animals recover from the pain condition. Both tramadol and morphine show efficacy on reversing beam walking impairments while amitriptyline does not. In the open field test, tramadol and diazepam does not reverse pain-related anxiety-like behaviour of CCI rats while gabapentin does, indicating that a pure analgesic or anxiolytic activity is not able to reverse the emotional-like abnormalities in neuropathic rats. In the object recognition test, Tramadol reversed the memory/attention deficit in CCI rats (data not shown). The method exampled below allows the investigation of the broader complex picture of pain and provides a useful pre clinical tool for the assessment of drug efficacy in restoring behavioural functionality and QoL in a rat pain model.

### 1.0 MATERIAL & METHODS

### 1.1 Animals

Male CD Sprague Dawley rats (CD) 200-250 g (Charles River, Margate, U.K.) are generally used for surgery. Rats are housed in group of three per cage under a 12 hour light/dark cycle with food and water available ad libitum. Each experiment is carried out with groups of 12 rats, randomising between CCI-, sham-operated and naïve rats during each trial. All procedures in this study are performed in accordance with the Home Office Animals (Scientific Procedures) Act 1986 and accordingly with our Project License, neuropathic rats and controls are sacrificed by schedule 1 method at 10 weeks post surgery.

### 1.2 NEUROPATHIC PAIN MODEL

The CCI of sciatic nerve is performed as previously described by Bennett and Xie (1988). Animals are anaesthetized with a 2% isofluorane/O₂ mixture maintained during surgery via a nose cone and the common sciatic nerve is exposed at the middle of the right thigh by blunt dissection through biceps femoris. Proximal to the sciatic trifurcation, about 7 mm of nerve is freed of adhering tissue and 4 ligatures (4-0 silk) are tied loosely around it with about 1 mm spacing. Ligatures are tied such that the circulation through the superficial epineural vasculature is retarded but not arrested. The incision is then closed in layers and the wound treated with topical antibiotics. In the sham-operated group an identical dissection is performed on the ipsilateral paw except the sciatic nerve is not ligated.

### 1.3 MEASUREMENT OF MECHANICAL-LIKE PAIN THRESHOLD

### 1.3.1 Static Allodynia

Animals are habituated for a couple of days to test cages prior to the assessment of mechano-allodynia. Static allodynia is evaluated by application of 9 calibrated von Frey filaments (Stoelting, Ill., USA.) to the plantar surface of hind paws in ascending order of force (1.0, 1.5, 2.0, 4.0, 5.0, 6.0, 8.0, 10.0 and 15.0 gram). Each von Frey hair is applied to the paw until a withdrawal response occurred or not more than 6 seconds. Once a withdrawal response is established, the paw is re-tested, starting with the next descending filament until no response occurred. The lowest amount of force required to elicit a response is recorded as paw withdrawal threshold (PWT, gram). Static allodynia is defined as animal responding equal or below the previously innocuous 4.0 gram von Frey hair (Field, et al, 1999, Pain; 83:303-11).

### 1.3.2 Dynamic Allodynia

Dynamic allodynia is assessed by lightly stroking the plantar surface of the hind paw with a cotton bud until a withdrawal response occurred. Care is taken to perform this procedure in fully habituated rats. At least three measurements are taken at each time point, the mean of which represents the paw withdrawal latency (PWL, sec). If no reaction is exhibited within 15 seconds the procedure is terminated and animals are assigned the cut off withdrawal time of 15 seconds. Dynamic allodynia is considered to be present if animals responded to the cotton stimulus within 8 seconds of stroking (Field, et al, 1999, Pain; 83:303-11).

### 1.4 BEHAVIOURAL TESTS

### 1.4.1 Locomotor Activity Test

The spontaneous locomotor activity of rats in a novel environment is monitored in a 35×20 cm Perspex chamber. The cage is equipped with two series photocells located at 2 and 15 cm above the floor (San Diego Instruments, CA, USA). Each animal is placed in the centre of the cage and the total locomotor activity (horizontal and vertical) is monitored every 5 minutes for a maximal time period of 30 minutes.

### 1.4.2 Beam Walking Test

The Beam walking apparatus consists of a 1.5 m long beam with a 2.5×2.5 cm square cross section, elevated 75 cm above the floor. The test is performed in dim light conditions (18 lux). A light source (520 lux) is placed at the start-end of the beam while a dark box at the other side (Goldstein & Davis, 1990, J Neurosci Methods; 31:101-107). Rats are trained over a period of 2 days to cross the beam. On the day of the test an additional training section is performed before the proper test trial was performed. The number of foot slips produced while a rat is crossing the beam are manually counted and a cut off of 10 foot slips is taken for those rats that do not cross or fall off the beam. Rats that cross the beam without using the ipsilateral paw are given a maximal number of foot slips.

### 1.4.3 Rota Rod Test

The rota rod test consists of 4 rotatable drums divided by flanges with a motor-driven drum which is capable of acceleration (Ugo Basile, Comerio, VA, Italy). For a given trial, a rat is placed on the rotating rod and the rotation speed is accelerated from 4 to 16 revolutions per minute (rpm) in 2 minutes. The time of maximal performance is set at 120 seconds (Voikar V et al, 2001, Physiol Behav., 2001; 72:271-81). Each animal received three training trials per day, at 1 hour intervals, for three consecutive days at the pre-test stage and three trials in a single day at each testing time post surgery. The latency to fall off the rod is represented as mean of the last three trials. Rats displaying a latency less than 80 seconds at the pre-test, are considered not to demonstrate normal performance and are excluded from the study.

### 1.4.4 Open Field Test

The spontaneous locomotor activity of rats in an open field is monitored for 30 minutes in a 70×70 cm dark arena (Prut ans Beizung, 2003, Eur J Pharmacol. 463:3-33). Each animal was placed in the centre of the arena and a video camera recorded movement of the animal. Four rats are recorded simultaneously in four different cages. Data are collected and analysed by Ethovision 3.0 software (Noldus IT, Netherdland) and the exploratory behaviour is expressed as number of entries in the central area of the arena (23×23 cm).

### 1.4.5 The Object Recognition Test

The Object Recognition test was performed as described by Ennaceur and Delacour (1988). The apparatus consisted of a black circular arena 55 cm in diameter with 50 cm black walls. The light intensity (60 Lux) was equal in the different parts of the arena. Two objects were placed in a symmetrical position about 10 cm away from the walls. We used 6 set of objects, different in shape and colour. The size of the objects was no bigger than twice rats dimension and were fixed in the arena floor thus could not be displaced by a rat.

Each animal was trained prior to testing; this involved handling and habituation of the rat to the arena for 5 minutes per session, twice a day for three consecutive days. A testing session comprises two trials. In the first trial (familiarisation phase) the apparatus contained two identical objects. The animal was placed in the centre of the arena facing the wall and allowed to explore two identical objects for 5 minutes. Subsequently, after a retention time of 4 and 24 hours, the animal was placed back in the apparatus for the second trial (sample phase). Now with two dissimilar objects, the familiar one (F) and a new one (N). In this phase, rats were allowed to explore the objects for 3 minutes. The distance moved in the arena and the time spent exploring each object during familiarization and sample phases were recorded manually and automatically, respectively by the videotracking system (Noldus Ethovision 3.0, Netherland).

Exploration was defined as follows: directing the nose towards the object at a distance of no more than 2 cm and/or touching the object with the nose. Sitting on the object was not considered exploratory behaviour. In order to avoid the presence of olfactory trails the objects and arena were always thoroughly cleaned. Moreover, none of the objects from the first trial were used in the second.

In the familiarization phase, any animal that explored the object, for less than 10 seconds or showing a preference for an object (difference in exploration time >10 seconds) is excluded from the study.

In the object recognition test, exploration is represented by the difference in exploratory time for the novel object over the familiar one (discrimination index, d=N−F). Data are represented as the mean of d±SEM of 8-16 rats per group and analyses by Mann Whitney t Test.

### 1.5 TESTS FOR PHYSICAL IMPAIRMENT

The rota rod method, as detailed above, was found to provide a good measure for locomotor impairment which is not associated with the specific intended pain condition, particularly in the CCI rat model, and which is in fact a result of a temporary physical impairment (for example muscle damage or denervation) which may be induced during procedures designed to produce the pain condition. For example the use of the rota rod method detected physical impairments displayed at 2 weeks post CCI surgery. Starting from 4 weeks post surgery, the locomotor performance is reversed indicating that the associated physical impairment is healed. To confirm these evidences, we tested the activity of morphine and tramadol in the rota rod in CCI rats and we found that both compounds did not improve rota rod performances of neuropathic rats (data not shown).

### 1.6 COMPOUNDS

Morphine (1 and 3 mg/kg, sc), Tramadol (10-100 mg/kg, PO), Amitriptyline (2-10 mg/kg, PO), gabapentin (30-100 mg/kg, PO) and mCPP (1 and 3 mg/kg, PO) are dissolved in physiological saline. Diazepam (1 and 3 mg/kg, IP) is suspended in 0.1% Tween 80. All drugs are supplied by Sigma Aldrich (Gillingham, UK) except gabapentin which is an in house synthesis.

### 1.7 DATA ANALYSIS

All the experiments are conducted in blind. When the experiment is carried out in more than one day and where technically possible, all groups occurred on each day with equal replication. Static allodynia is expressed as median [LQ; UQ] while the number of foot slips in the beam walking test as mean±SEM both parameters are analysed by Mann Whitney U test. In the object recognition test, exploration is represented by the difference in exploratory time for the novel object over the familiar one (discrimination index, d=N−F). Data are represented as the mean of d±SEM of 8-16 rats per group and analyses by Mann Whitney t Test. For all the others studies, data are expressed as mean±SEM and analysed by ANOVA.

### 1.8 RESULTS

### 1.8.1 General Health and Sensory Scores

After surgery animals appeared well groomed and most of the time CCI rats kept the injured paw in a guarded position. In ten weeks of observation rats gained weight normally and no differences between CCI-, sham-operated and naïve rats are observed. When in a sitting or standing position animals often kept the paw off the ground in a guarded position next to the flank and often seen licking the injured paw. This behaviour is certainly more frequent in the first two weeks post surgery while at 8 weeks most of rats did not show guarded position and their ambulation is almost like the control groups. To allow animals to recovery from surgery, all behavioural test are carried out starting from two weeks post nerve injury, which corresponds to the time of onset of pain (Field M J et al, 1999).

The chronic constriction injury (CCI) of sciatic nerve produced a long lasting decrease in the pain-like threshold in rats (Bennett and Xie, 1988; Field et al, 1999). Before surgery the ipsilateral and contralateral paws respond to high noxious stimuli only (≧8 g and ≧9 seconds). Thus the mean in the ipsilateral paw for static mechanical pain-like threshold is 15 gram [5; 0] while for dynamic mechanical pain-like threshold is 12.6±0.6 seconds. The contralateral paw maintained a similar value for the entire time course with no significant differences between naïve, sham- and CCI-operated profiles (data not showed) and ipsilateral value of sham-operated and naïve.

At two weeks post surgery the static and dynamic pain-like threshold measured in the ipsilateral paw are strongly reduced (data not shown). About 90% of rats showed a pain-like threshold ≦4 gram or 9 seconds for static and dynamic allodynia, respectively. The mean value of CCI rats is significantly different from controls in both sub-types of pain (p<0.01). Static allodynia is 4 gram [0; 0] vs 10 gram [2; 0] while dynamic is 4.4±0.7 vs 10.9±0.8 seconds for naïve rats. The pain-like threshold in CCI rats remained consistently stable up to 6 weeks and only at 8 weeks post surgery the percentage of rats showing an allodynic-like behaviour decreases. On the overall, the pain-like threshold on the ipsilateral side of CCI rats at 8 and 10 weeks post nerve injury is not statistically different from controls and no different from the pre surgery value.

### 1.8.3 Locomotor Activity Test

The locomotor activity of CCI-, sham-operated and naïve rats is recorded starting from 2 weeks post surgery. The total movement in a new environment is measured in all groups for 30 minutes (FIG. 1A). The CCI rats showed a significant decrease in the locomotor activity at 2 weeks post surgery only (318±26 vs 438±41 and 417±26 counts for sham-operated and naïve, respectively). At 4 weeks post surgery CCI rats spontaneously explore a new environment as controls and the locomotor activity of all groups is not statistically different. Further studies showed that the decrease in spontaneous locomotor activity of CCI rats is also present at 1 weeks post surgery (data not showed) but is never seen after 2 weeks post injury.

### 1.8.4 Rota Rod Test

Fourteen days post nerve injury the rat's coordination performances are evaluated by the accelerated rota rod (FIG. 1B). The profile of naïve and sham-operated rats is not significantly different during the testing period. Both groups displayed a latency to fall not statistically different from their corresponding baseline at two weeks (99±7 vs 112±3 seconds and 102±5 vs 111±5 seconds, for sham and naïve respectively). Nerve injured rats instead, at this time point displayed motor deficits in the rota rod task. The latency to fall decreased by 59% compared to the baseline (p<0.01) and the percentage of rats underperformance (mean latency<80 seconds) is 67% while 33% and 8% for naïve and sham groups, respectively. Two weeks later, the percentage of rats able to remain on the rod for more than 80 seconds increased and only 33% of CCI group are under performance (17% for both naïve and sham; NS). Further studies, demonstrated that the physical disability of CCI rats in the rota rod test is showed also at 1 week post surgery. Selected rats (mean performance<80 seconds) treated with analgesic doses of morphine or tramadol did not showed improvement in the rota rod test (data not shown).

### 1.8.5 Beam Walking Test

A week before surgery, rats are trained, for two days, to cross the entire length of the beam in less than 10 seconds (5.0±0.2 seconds) and with one or no foot slips (0.5±0.1; FIG. 1C). At two weeks post surgery, we monitored the level of motor coordination in CCI rats and controls by testing again their ability to cross the beam. Both naïve and sham groups maintained a normal ambulation for the entire time course. They crossed the beam in less than 5 seconds and did not display significant changes in the number of foot slips compared to the baseline. No control rats fell off the beam or showed freezing behaviour. On the contrary the neuropathic rats, at 14 days after nerve ligation, are unable to cross the beam correctly; they showed a significant increase in the number of foot slips produced (14.2±1.8 vs 4.0±0.4 for sham-operated group). About 42% of neuropathic rats fell off the beam and 1 did not cross at all. The impairments lasted for 6 weeks after nerve damage; at this time the number of foot slips in the CCI group are still significantly different from controls (2.4±0.9 vs 0.3±0.1 for sham-operated group; p<0.01). At 7 weeks after nerve injury, the performance CCI rats improved and the number of foot slips is not different from controls.

Further studies, demonstrated that the physical disability of CCI rats in the beam walking test is also present at 1 week post surgery (data not shown).

Starting from 3 weeks post surgery, CCI rats are selected based on their performances. Only those showing a number of foot slips of 2 or above are selected and used for the pharmacological validation of the test. A group of naïve CD rats are treated with vehicle and used as positive controls. Morphine (1 and 3 mg/kg), tramadol (30-100 mg/kg) and amitriptyline (2 mg/kg) are administered subcutaneously (s.c.), orally (p.o.) and intraperitoneally (i.p.), respectively and rats are tested at 30 minutes, 1, 2 and 3 hours post dosing (FIG. 2). Morphine significantly decreased the number of foot slips compared to baseline (3±1 and 2±1 vs 6±1 and 5±1 for 30 minutes and 1 hour, respectively at the higher dose; p<0.05). Although the lower dose of 1 mg/kg improved the rats performance in the beam walking test the value is not statistically different from the control value (FIG. 2A).

Tramadol dose dependently improves in the ability of CCI rats to cross the beam. At 1 hour post drug administration, rats treated with the higher dose, showed a significant reduction in the number of foot slips (1±1 vs 6±1 of CCI vehicle-treated group; p<0.01). Apparently, also the dose of 30 mg/kg improved the performance of rats, however data are not significantly different from CCI controls (FIG. 2B).

Amitriptyline did not improve neuropathic rats performance in the beam walking task (FIG. 2C) even at 20 mg/kg (data not shown).

### 1.8.6 Open Field Test

To measure the anxiety-like behaviour in CCI rats the open field test is carried out in normal light conditions (100 Lux). Higher intensity induced an anxiety-like behaviour such as thigmotaxis and freezing in control naïve and did not help to differentiate between CCI rats, naïve and sham. To confirm that the environmental conditions we set up, could let us measure an anxiety-like behaviour, the activity of the anxiogenic compound mCPP was tested in CD naïve rat.

mCPP (1-3 mg/kg) intraperitoneally (i.p.) produced a dose-dependent decrease in the exploratory activity (e.g. number of entries) of rodent (FIG. 3). The exploratory behaviour in the centre of the arena is significantly reduced at the higher dose (5±2 vs 13±3 of vehicle-treated animals). Seventy-five percent of rats, compared to 16% in the control group, explored the centre less than 6 times.

A group of 60 rats underwent CCI of the sciatic nerve and starting from two weeks post surgery, were tested in the open field task (FIG. 4A). Injured rats showed a decrease in the exploratory activity from 2 to 6 weeks post nerve damage. The number of entries in the central area is strongly reduced with a mean value similar to that showed by naïve rats treated with mCPP (5±1 vs 5±2, respectively). At 9 weeks post surgery the exploratory activity increased and was significantly different from 6 weeks data (9±1; p<0.05).

We observed that in this population of injured rodents almost 50% of them still showed allodynic pain-like threshold at 9 weeks post surgery. Therefore we divided animals in two sub groups based on the pain-like threshold (static allodynia) at 9 weeks and we found that early recovery rats explored the open field significantly less at 6 weeks post surgery compared to the late recovery group which still showed anxiety-like behaviour at this time point (FIG. 4B).

For a pharmacological validation of the model, CCI rats with a poor exploratory activity (entries≦6) are pre selected in the open field and a week later treated with diazepam (1 and 3 mg/kg, i.p.), tramadol (30 mpk, p.o.) or gabapentin (30-100 mg/kg, p.o.). Naïve CD vehicle-treated rats are used as positive control. As shown in FIG. 5A, diazepam and tramadol did not pain-related anxiety-like behaviour of CCI rats even at higher doses such as 3 mg/kg and 100 mg/kg, respectively (data not shown). On the contrary, gabapentin improved the exploratory activity of neuropathic rats in a dose dependent manner (FIG. 5B). Both doses increased the number of entries in the centre of the open field compared to CCI vehicle-treated group, however only 100 mg/kg completely reversed the anxiety-like behaviour of injured rats (12±2 vs 14±2 for naïve vehicle-treated group; NS).

The present example demonstrates that the chronic constriction injury (CCI) rat model, which is a well-established model of neuropathy, is a powerful tool in the method for selecting compounds in the early stages of compound search.

### 1.8.7: The Object Recognition Test

Since we demonstrated that CCI rats develop major motor dysfunction in the first two weeks post surgery and that sham-operated are substantially not different from naïve, we proceeded examining the memory function at 3 weeks post surgery and, as control group, we used non operated rats in respect to the UK Home Office Animal Act 1986. Thus, CCI rats and naïve were tested at 3, 5, 6 and 8 weeks post injury.

During the familiarization trial most of naïve and CCI rats explored both objects for more than 10 seconds showing no preferences for one object over the other (difference in exploration between the two objects was −1.6±1.1 seconds and 2.7±1.2 seconds for naïve and CCI respectively at 3 week post surgery). In both groups from 30 to 50% of rats were excluded every week as d>10 seconds and re-tested the following week.

Rat's ability to recognize the new object was tested at 4 and 24 hours after the familiarization phase (FIGS. 6 A and B). At both time points naïve rats consistently explore the novel object more than the familiar one (d=5.2±3.4 and 17.5±2.8 seconds as max and min value over the time course at 4 and 24 hours). On the contrary, the CCI group found difficulties in discriminate the two objects. At 3 and 5 weeks post nerve damage, the performances of CCI were statistically different from controls at both 4 and 24 hours post familiarization phase. Six weeks post injury, the ability to recognize the new object was not significantly different from controls at 4 hours. However, at 24 hours neuropathic rats still failed to explore the new object, indicating a dysfunction in the neurophysiology of memory. No differences were observed at 8 weeks after surgery. These changes can be related to cognitive impairments only as the total exploration (the sum of the time spend exploring the new object and the familiar object) was slightly different at 5 weeks only and consistent between CCI and naïve for the rest of the time course (FIG. 7). Tramadol was tested in this assay to establish whether the cognitive dysfunction could have been reversed by a standard clinically active analgesic compound. Nerve injured and naïve rats were tested in the familiarization phase and only animals that explored equally the two identical objects (d<10 seconds) were selected for the pharmacological study. At 3.5 hours post familiarization, CCI rats were treated with saline or Tramadol (100 mg/kg, po) while naïve rats were used as positive control and treated with saline (1 ml/kg, PO). Thirty minutes later, the second phase of object recognition test was performed. As showed in FIG. 8, Tramadol reversed the cognitive dysfunction of neuropathic rats showing a significant increase in the discrimination index compared to vehicle-treated CCI rats (P<0.01). Neuropathic rats showed a stronger interested for the novel object over the familiar suggesting that an analgesic treatment can improve cognitive deficits developed following nerve injury.

### 1.9 FIGURES

FIG. 1: Development of motor deficit in CCI rats. Locomotor activity (A), rotarod (B) and beam walking tests (C) are carried out naïve (white square), sham- (white triangle) and CCI-operated (black circle) rats. Data are the mean±SEM of 12 animals per group. *p<0.05 and **p<0.01 vs naïve group (ANOVA) for latency and counts (A and B). **p<0.01 vs naïve group (Mann Whitney U test) for foot slips (C).

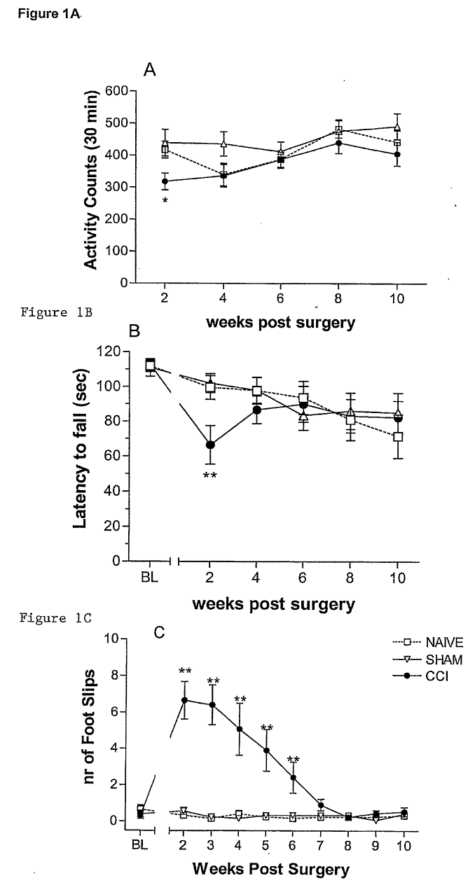

FIG. 2: Effect of morphine (A), tramadol (B) and amitryptiline (C) in the beam walking test in CCI rats. Morphine is given at 1 and 3 mg/kg, sc, Tramadol at 30 and 100 mg/kg, po while Amitryptiline at 2 mg/kg, po in CCI rats. A group of naïve (black square) and CCI (white square) treated with vehicle are used as positive and negative controls, respectively. Data are the mean±SEM of 7-10 rats per group. *p<0.05 and **p<0.01 vs CCI vehicle-treated group (Mann Whitney U test).

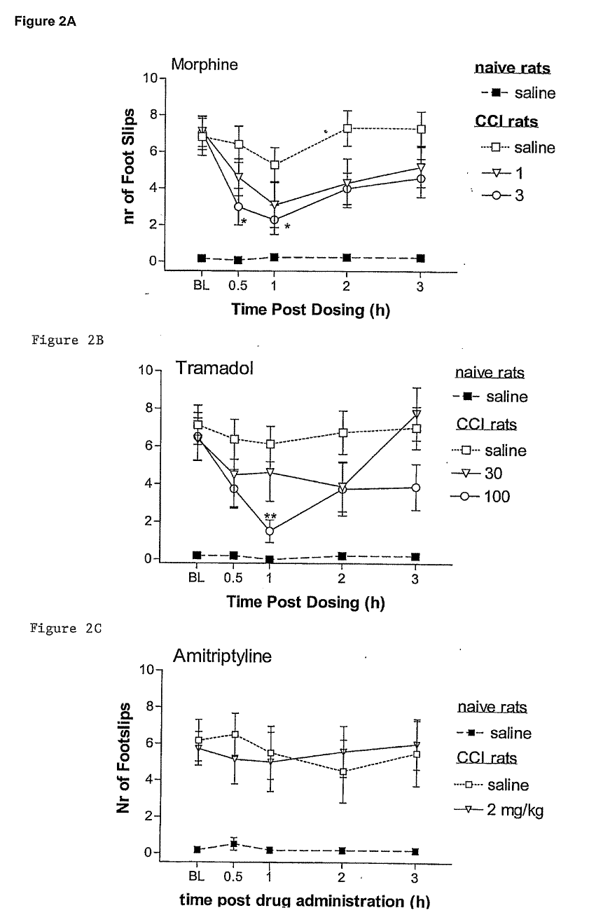

FIG. 3: Comparison of mCPP effect in naïve rats in the open field test. Exploratory behaviour is defined as the number of entries in the central area of the arena. Data are the mean±SEM of 10 naïve rats per group *p<0.05 vs vehicle treated naïve rats (ANOVA).

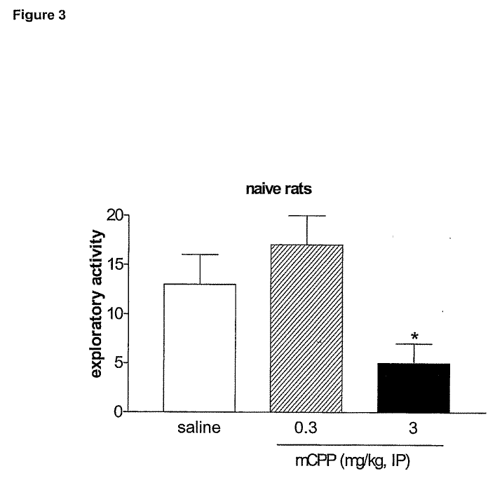

FIG. 4: Time course of anxiety-like behaviour of CCI rats (A) and comparison between early and late recovery injured rats (B). Animals have been divided in two groups based on their pain-like threshold at 9 weeks post surgery. Late recovery are a group of CCI rats showing static mechanical allodynia at 9 weeks (PWT≦4 g). Injured animals with a PWT>4 g are classified are early recovery. Data are the mean±SEM of 26-34 rats per group. *p<0.05 vs early recovery group at 6 weeks post surgery (ANOVA).

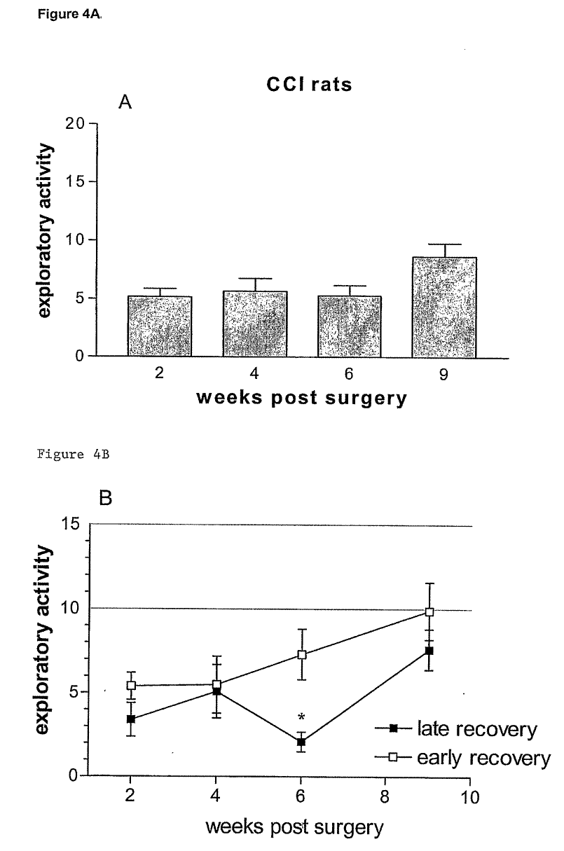

FIG. 5: Effect of diazepam, tramadol (1 mg/kg, IP and 30 mg/kg, PO, respectively; (A) and gabapentin (30 and 100 mg/kg, PO; (B) in the open field test. Diazepam and Tramadol are given 30 minutes while gabapentin at 1 hour before the test. Exploratory behaviour is defined as the number of entries in the central area of the arena. A group of naïve (white bar) and CCI (grey bar) treated with vehicle are used as positive and negative controls, respectively. Data are the mean±SEM of 7-10 rats per group. **p<0.01 vs vehicle-treated naïve rats, ##p<0.01 vs vehicle treated-CCI rats

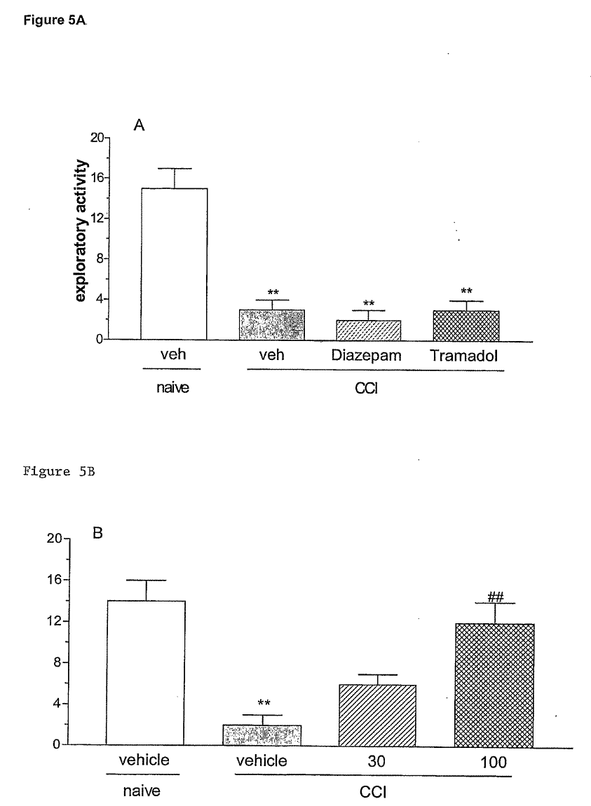

FIG. 6: Development of cognitive impairments in CCI rats. Naïve (white columns) and CCI (black columns) were tested in the object recognition task at 4 hours (A) and 24 hours (B). Graph represent the second phase of the test. Data are the mean±SEM of 9-16 rats per group. *p<0.05 and **p<0.01 vs naïve group (Mann Whitney T Test)

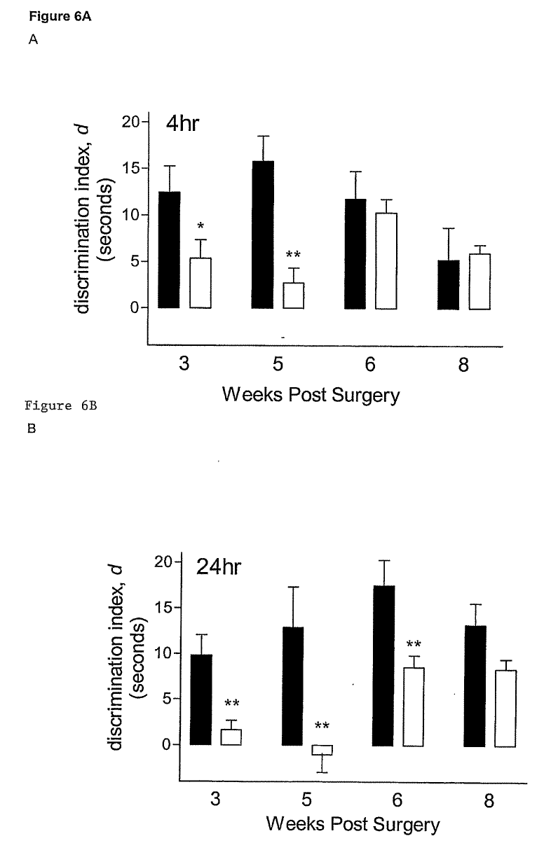

FIG. 7: Total exploratory time of both objects during the test phase

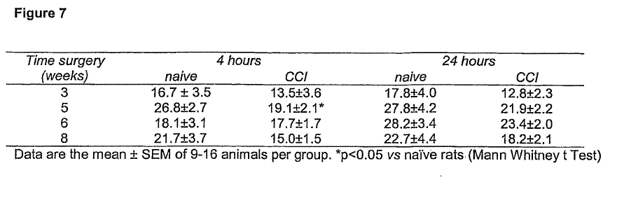

FIG. 8: Effect of Tramadol in the object recognition test in CCI rats. Tramadol (100 mg/kg, PO) or saline are given 3.5 hours post familiarization phase and rats are tested in the second phase 30 minutes post treatment. Naïve CD rats treated with saline are used as positive control. Data are the mean±SEM of 7 rats per group. **p<0.01 vs naïve vehicle-treated group and #p<0.05 vs CCI saline treated group (Mann-Whitney analysis).

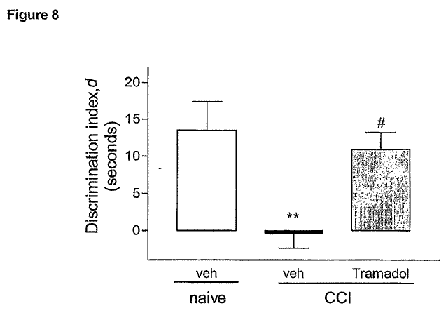

### REFERENCES

2. Goldstein L B, Davis N J. Beam-walking in rats: studies towards developing an animal model of functional recovery after brain injury. J Neurosci Methods 1990; 31:101-107.
3. Niv D, Kreitler seconds. Pain and Quality of Life. Pain Practice, 2001; 1:150-161.
4. Skevington S M. Investigating the relationship between pain and discomfort and quality of life, using the WHOQOL. Pain, 1998; 76:395-406.
5. Bennett G J, Xie Y K. A peripheral mononeuropathy in rat that produces disorders of pain sensation like those seen in man. Pain, 1988; 33:87-107.
6. Field M J, Bramwell seconds, Hughes J, Singh L. Detection of static and dynamic components of mechanical allodynia in rat models of neuropathic pain: are they signalled by distinct primary sensory neurones? Pain, 1999; 83:303-11.
7. Voikar V, Koks seconds, Vasar E, Rauvala H. Strain and gender differences in the behavior of mouse lines commonly used in transgenic studies. Physiol Behav., 2001; 72:271-81.
8. Vrinten D H, Hamers F F. ‘CatWalk’ automated quantitative gait analysis as a novel method to assess mechanical allodynia in the rat; a comparison with von Frey testing. Pain. 2003; 102:203-209.
9. Prut L, Belzung, C. The open field as a paradigm to measure the effects of drugs on anxiety-like behaviors: a review. Eur J. Pharmacol. 2003; 463:3-33.
10. Gureje O, Von Korff M, Simon G E, Gater R. Persistent pain and well-being, JAMA 1998; 280:147-151.
11. McWilliams L A, Cox B J, Enns M W. Mood and anxiety disorders associated with chronic pain: an examination in a nationally representative sample. Pain, 2003; 106:127-33.
12. Millecamps M et al, 2003 abs EFIC Prague 2003
13. Kontinen V K, Kauppila T, Paananen seconds, Pertovaara A, Kalso E. Behavioural measures of depression and anxiety in rats with spinal nerve ligation-induced neuropathy. Pain 1999; 80:341-346
14. Schrijver N C, Bahr N I, Weiss I C, Wurbel H. Dissociable effects of isolation rearing and environmental enrichment on exploration, spatial learning and HPA activity in adult rats. Pharmacol Biochem Behav 2002; 73:209-224.
15. Singh L, Field M J, Ferris P, Hunter J C, Oles R J, Williams R G, Woodruff G N. The antiepileptic agent gabapentin (Neurontin) possesses anxiolytic-like and antinociceptive actions that are reversed by D-serine. Psychopharmacology (Berl). 1996; 127:1-9.
16. Mease P J et al, abs EFIC, Prague 2003
17. Backonja M, Beydoun A, Edwards K R, Schwartz S L, Fonseca V, Hes M, LaMoreaux L, Garofalo E. Gabapentin for the symptomatic treatment of painful neuropathy in patients with diabetes mellitus: a randomized controlled trial. JAMA, 1998; 280:1831-1836.
18. Rowbotham M, Harden N, Stacey B, Bernstein P, Magnus-Miller L. Gabapentin for the treatment of postherpetic neuralgia: a randomized controlled trial. JAMA, 1998; 280:1837-1842.
19. Silver A. Haeney M. Vijayadurai P. Wilks D. Pattrick M. Main C J. The role of fear of physical movement and activity in chronic fatigue syndrome. Journal of Psychosomatic Research. 2002; 52: 485-493.
20. Geisser M E. Haig A J. Theisen M E. Activity avoidance and function in persons with chronic back pain. Journal of Occupational Rehabilitation. 2000, 10: 215-227.
21. Kim S H, Chung J M. An experimental model for peripheral neuropathy produced by segmental spinal nerve ligation in the rat. Pain. 1992; 50:355-363.
22. Feeney D M et al, 1982
23. Maier N. R. F. (1935) J. Comp. Neurol, 61: 395-405;
24. Vowles K E, Gross R T. Work-related beliefs about injury and physical capability for work in individuals with chronic pain. Pain. 2003; 101:291-298.
25. Schrijver N C, Bahr N I, Weiss I C, Wurbel H. Dissociable effects of isolation rearing and environmental enrichment on exploration, spatial learning and HPA activity in adult rats. Pharmacol Biochem Behav. 2002; 73:209-24.
Carey M P, Fry J P. Evaluation of animal welfare by the self-expression of an anxiety state. Lab Anim. 1995; 29:370-379.

1. An assay comprising the following steps:
(a) providing a non human test animal which has been arranged to experience a pain condition,
(b) determining the degree to which the test animal experiences pain,
(c) determining whether the test animal experiences pain or physical impairment not associated with the pain condition and selecting the test animal if the pain is associated predominantly with the pain condition,
(d) determining a performance score for the selected test animal in one or more behavioural tests,
(e) determining whether the selected test animal demonstrates an increase, decrease or no change in performance score compared to the performance score obtained for a control animal.

2. A modification of the assay of claim 1 comprising the following steps:
(a) providing a non human test animal which has been arranged to experience a pain condition,
(b) determining the degree to which the test animal experiences pain,
(c) determining whether the test animal experiences pain or physical impairment not associated with the pain condition and selecting the test animal if the pain is associated predominantly with the pain condition,
(d) determining the performance score for the selected test animal in one or more behavioural tests,
(e) administering a test compound to the selected test animal,
(f) determining whether there is an increase, decrease or no change in the degree to which the selected test animal continues to experience pain,
(h) redetermining a performance score for the selected test animal in the one or more behavioural tests,
(e) determining whether the selected test animal demonstrates an increase, decrease or no change in performance score.

3. The assay according to claim 1 or claim 2 wherein the pain condition is selected from, neuropathic pain, inflammatory pain, nociceptive pain, musculo-skeletal pain, on-going pain, chronic pain, central pain, heart and vascular pain, head pain, orofacial pain.

4. The assay according to claim 3 wherein the pain condition is chronic pain.

5. The assay according to claim 3 wherein the pain condition is neuropathic pain.

6. The assay according to claim 2 wherein the non human test animal has been arranged to experience a pain condition by chronic constriction injury.

7. The assay according to claim 1 or claim 2 wherein whether the test animal experiences pain or physical impairment not associated with the pain condition is determined by performance of a locomotor impairment test.

8. The assay according to claim 7 wherein the locomotor impairment test is the rota rod test.

9. The assay according to claim 1 or claim 2 wherein the behavioural test(s) is/are designed to measure one or more of cognitive function, social well-being, emotional well-being or physiological well-being.

10. The assay according to claim 1 or claim 2 wherein the behavioural test(s) is/are designed to measure one or more of; learning ability, memory, social interaction, exploratory behaviour, motivation, anxiety, depression, spontaneous locomotion and activity, sexual behaviour, quality of sleep, change in body weight.

11. The assay according to claim 1 or claim 2 wherein the behavioural test is selected from, a locomotor activity test, a beam walking test, a rota rod test, an open field test, object recognition test.

12. The assay according to claim 1 or claim 2 wherein the test animal is trained and/or habituated in the environment or performance of the behavioural test prior to the determination of the performance score.
